(19) United States
(12) Patent Application Publication
(10) Pub. No.: US 2016/0367450 A1
(43) Pub. Date: Dec. 22, 2016

(54) COSMETIC COMPOSITIONS

(71) Applicant: Cosmetics Limited, Nassau (BS)

(72) Inventor: Robin Parker, Bath (GB)

(21) Appl. No.: 15/183,732

(22) Filed: Jun. 15, 2016

(30) Foreign Application Priority Data
Jun. 16, 2015 (GB) 1510592.7

(51) Int. Cl. A61K 8/27; A61K 8/36; A61K 8/19

(52) U.S. Cl. CPC A61K 8/27; A61K 8/19; A61K 8/36

(57) ABSTRACT

A cosmetic product contains, in one or more cosmetic components each comprising a cosmetic base, zinc powder and/or copper powder, and an organic acid. The zinc powder and/or copper powder are encapsulated with a hydrophobic encapsulation which prevents the organic acid coming into contact with the zinc powder and/or copper powder. The encapsulating material may be a brittle waxy material such as one comprising stearyl palmitate. The organic acid may be selected from 10-hydroxydecanoic acid, acetyl salicylic acid, alphahydroxy acid, glycolic acid, succinic acid, lactic acid, ferulic acid and hyaluronic acid and mixtures thereof. The composition may be used in a method of applying a cosmetic product for the skin. The composition may also be used in a method of improving the cosmetic appearance of the skin by applying the cosmetic product to the surface of the skin and massaging the skin to break the encapsulation to allow the zinc and/or copper powder to react with the organic acid.

### CROSS-REFERENCE TO RELATED APPLICATIONS

This application claims priority to Great Britain application No. 1510592.7 which was filed on Jun. 16, 2015.

### FIELD OF INVENTION

The present invention relates to cosmetic compositions. In particular to compositions for increasing the metabolic rate of skin cells and, thereby, improving the appearance of the skin.

### BACKGROUND

Cosmetic moisturisers, or emollients are chemical compositions used to make the skin and, in particular, the epidermis of skin, softer and more hydrated, to give the skin an improved tone and/or texture and to counteract the effects of aging, such as wrinkles.

Various types of moisturiser are available which seek to increase a skin's hydration, by reducing evaporative loss of moisture or by replenishing moisture. More advanced cosmetic compositions may additionally seek to restore deficient nutrients or other depleted skin components in order to improve skin function and appearance.

A cosmetic composition may contain a “skin energizer” which seeks to restore skin tone by boosting the metabolism of the skin cells and, in particular, of the epidermal cells. Higher metabolic rates may result in increased rates of structural protein synthesis and increased rates of cell renewal, both of which may slow the visible signs of skin aging.

Various cosmetic ingredients able to increase cell metabolic rate are known. These include Sepitonic M3™ manufactured by Seppic S. A., Paris, France. Sepitonic M3™ is a proprietary composition comprising magnesium aspartate, zinc gluconate and copper gluconate. Greentech of Saint Beauzire, France sells a different product comprising glycerin and mannosyl glucuronic acid polyglucans under the trade name Somitine™. Both of these products are supported by experimental evidence showing an ability to increase the metabolism of skin cells when applied topically and thereby improve the appearance of the skin.

Various cosmetic products and services use the techniques of electrostimulation and iontophoresis whereby the skin is stimulated by applying a current to the skin in order to electrically stimulate skin cells in order to increase their metabolism and thereby skin appearance. It is also thought that the current can be used to encourage the migration of beneficial charged compounds into the skin and potentially non-beneficial charged compounds out of the skin. Such treatments require the use of electrostimulation apparatus and are typically carried out by professional therapists at a spa or on the premises of a cosmetic therapy provider. There is a need for electrostimulation of skin to be provided to users as a “use at home” cosmetic product so that it may be used as an alternative to professionally administered therapies or for use between professionally administered therapy sessions.

### SUMMARY OF INVENTION

According to the first aspect of the invention there is provided a cosmetic product comprising in one or more cosmetic components each comprising in a cosmetic base:
zinc powder and/or copper powder; and
an organic acid,
   wherein the zinc powder and/or copper powder are encapsulated with a hydrophobic encapsulation which prevents the organic acid coming into contact with the zinc powder and/or copper powder.

According to the second aspect of the invention, there is provided a method of applying a cosmetic product for the skin comprising:
applying a first component of the product to the skin;
applying a second component of the product to the skin;
massaging the skin to mix the first and second components together, characterized in that one or both components contains an organic acid, and one or both components contain zinc powder and/or copper powder which has been encapsulated with a hydrophobic encapsulation, such that when the skin is massaged to mix the first and second components together, the hydrophobic encapsulation is ruptured allowing the zinc powder and/or copper powder to come into contact with the organic acid.

According to the third aspect of the invention, there is provided a method of improving the cosmetic appearance of skin by boosting metabolic rate of epidermal cells comprising:
applying one or more compositions to the surface of the skin;
wherein the one or more compositions collectively comprise an organic acid in an aqueous cosmetic base and zinc powder and/or copper powder, wherein the zinc powder and/or copper powder is encapsulated in a hydrophobic encapsulation such that the zinc powder and/or copper powder is wholly or partly prevented from reacting with the organic acid;
massaging the skin surface to break the encapsulation and allow the zinc powder and/or copper powder to react more fully with the organic acid.

### DETAILED DESCRIPTION

According to the first aspect of the invention there is provided a cosmetic product comprising in one or more cosmetic components each comprising in a cosmetic base:
zinc powder and/or copper powder; and
an organic acid,
wherein the zinc powder and/or copper powder are encapsulated with a hydrophobic encapsulation which prevents the organic acid coming into contact with the zinc powder and/or copper powder. It is to be understood that when the product comprises both zinc powder and copper powder they are separately encapsulated such that when encapsulated they are not in contact with each other.

According to the second aspect of the invention, there is provided a method of applying a cosmetic product for the skin comprising:
applying a first component of the product to the skin;
applying a second component of the product to the skin;
massaging the skin to mix the first and second components together, characterized in that one or both components contains an organic acid, and one or both components contain zinc powder and/or copper powder which has been encapsulated with a hydrophobic encapsulation, such that when the skin is massaged to mix the first and second components together, the hydrophobic encapsulation is ruptured allowing the zinc powder and/or copper powder to come into contact with the organic acid.

It is noted that according to the first aspect of the invention the product may comprise a single component (although optimally multiple components may be used) whereas the second aspect requires at least two components and a subsequent mixing of the two components. The use of two or more components allows the organic acid and encapsulated metal powder(s) to be kept apart until mixing. However, this is not essential because the hydrophobic encapsulation keeps the two ingredients apart until rupture. This allows the two ingredients to be provided in a single component. When that is the case, the use of two components is primarily so that the user can be instructed to massage them onto the skin to mix them together whereby the massaging step is required for the encapsulation to rupture.

Optionally the two components may be coloured differently so that the extent of mixing between them can be observed as an encouragement to the user to massage sufficiently for rupture of the encapsulation to sufficiently occur.

According to the third aspect of the invention, there is provided a method of improving the cosmetic appearance of skin by boosting metabolic rate of epidermal cells comprising:
applying one or more compositions to the surface of the skin;
wherein the one or more compositions collectively comprise an organic acid in an aqueous cosmetic base and zinc powder and/or copper powder, wherein the zinc powder and/or copper powder is encapsulated in a hydrophobic encapsulation such that the zinc powder and/or copper powder is wholly or partly prevented from reacting with the organic acid;
massaging the skin surface to break the encapsulation and allow the zinc powder and/or copper powder to react more fully with the organic acid.

The boost to metabolism may be assessed by any convenient method including the glucose consumption assay described herein. The cosmetic appearance of skin may be assessed by any convenient method for example by use of a wrinkle measuring apparatus.

According to certain embodiments the compositions may comprise any of the ingredients disclosed elsewhere herein, for example the metal powders, encapsulation and identity of organic acid(s) may be as described herein in reference to other aspects of the invention.

Cosmetic Product

According to all aspects of the invention, the cosmetic product may comprise a single component cosmetic product or a multicomponent cosmetic product. A multicomponent cosmetic product may comprise multiple (for example 2, 3 or 4) components packaged together, sold together or marketed for use together. For example, it may comprise 2, 3 or 4 components each of which may be packaged in their own bottle, tube, tub, pot or dispenser which are then packaged into a box or other outer packaging to be sold together. Alternatively, each component may be packaged into a separate compartment of a multi-compartment dispenser, for example into separate juxtaposed compartments of a pump dispenser. According to certain embodiments the multicomponent cosmetic product may comprise two or more of the following components:

A—Exfoliating Components

Components which assist in the removal of the oldest dead skin cells on a skin's outermost surface in order to improve the skin's appearance and /or surface electrical conductivity so as to increase the efficiency of electro-stimulation. An exfoliating component may comprise a cosmetic base and abrasive particles including, micro beads, crushed apricot or almond shells, crystals, pumice powder or other abrasive particles. Alternatively an exfoliating component may be an exfoliating tool such as a pumice stone, a sponge, emery, loofah or brush, cloth, paper or adhesive sheet. Alternatively, an exfoliating component may contain a chemical exfoliant such as salicylic acid, glycolic acid, citric acid, malic acid or alpha or beta hydroxy acids. In some embodiments the exfoliating component may combine both chemical and mechanical exfoliating agents. According to certain embodiments the encapsulated zinc and/or copper powders are not provided in Component A, although the invention includes embodiments where the encapsulated zinc and/or copper powders are provided in an exfoliating component.

B—Energising Component

Typically this component comprises an aqueous cosmetic base. It may also contain one or more further ingredients designed to energise the skin or otherwise improve the skin's appearance. According to certain embodiments, this component may contain an energiser composition such as magnesium aspartate, zinc gluconate and copper gluconate (for example a composition sold under the Sepitonic M3™ brand and/or a composition sold under the Somitine™ brand). The encapsulated zinc powder and/or encapsulated copper powder are preferably provided in this component. The organic acid is optionally provided in this component although in some embodiments the organic acid is provided in (one of) the other component(s). Provision of encapsulated zinc powder, encapsulated copper powder and/or organic acid may optionally and additionally be made in other components.

C—Day Cream

This is a moisturising cream intended for use during the day. In a cosmetic base it may contain various ingredients to improve skin moisturisation and appearance including salts, oils, fats, acids and fragrances. According to certain embodiments it contains sea salt, lactic acid, ferulic acid (as a barley extract), succinic acid, amber fragrance (which contains succinic acid), a rejuvenating plant peptide extract, cranberry seed oil, shea butter, coconut oil, hyaluronic acid, almond oil. The encapsulated zinc powder and/or encapsulated copper powder may be provided in this component although more preferably they are provided in component B when that component is present. The organic acid is preferably provided in this component.

D—Night cream

This is a moisturising cream intended for use during the night. It typically is designed to provide more intensive moisturisation than an equivalent day cream and although it may contain many ingredients in common with the day cream described above, it typically contains moisturisation-providing components such as shea butter at higher levels than the day cream. Additional components may optionally include hydrogenated vegetable fatty acids such as the hydrogenated sunflower fatty acids sold in the Simulskin™ product and Sepitonic M3™ and jojoba oil. The encapsulated zinc powder and/or encapsulated copper powder may be provided in this component, although more preferably they are provided in component B, when that component is present. The organic acid is preferably provided in this component.

According to certain embodiments the cosmetic product consists of component B, C or D only. According to other embodiments, the cosmetic product consists of components B and C (as a “day product”) or B and D (as a “night product”) or as B, C and D (as a product for dual day and night use).

If an exfoliating component is supplied the product may comprise A, B and C; A, B and D; or A, B, C and D.

Packaging

The different components may be packaged into separate containers and combined to a single product according to the invention by being packaged into an outer bag or box, for example a cardboard carton, optionally with a suitable card or plastic insert. Containers suitable for use in accordance with the invention include tubes, tubs, pots and pump dispensers. According to certain embodiments the components are packaged into a container having a pump dispenser. According to certain embodiments each component of a multicomponent product is packaged into a separate compartment of a multi-compartment container, for example a multicompartment pump dispenser, such that the components are conveniently kept together. Each component is typically dispensed separately, for example by the provision of a separate pump for each compartment of the dispenser. Alternatively, where two or more components are intended to be stored separately but used together, separate pumps may be activated by a common mechanism such that multiple components are dispensed simultaneously.

Cosmetic Bases

Cosmetic bases for use in accordance with the invention may be creams or gels used to provide a pleasing appearance and feel and to carry the other ingredients. Typically such bases are oil in water emulsions or water in oil emulsions or hydrogel formulations.

Zinc Powder

Zinc powder for use in accordance with the invention is typically metallic zinc having a particle size of 150 μm, for example of less than 100 μm or less than 50 μm, less than 25 μm, less than 20 μm, less than 10 μm less than 5 μm. The above particle sizes refer to diameters of spherical particles. Where the particles are not spherical the figure refers to the diameter of a volume equivalent sphere. In any sample of powder there will be some size variability and so the above size criteria are to be understood as being met if greater than 95% of the zinc particles (preferably by mass) meet the size criteria stated. Although the powder is encapsulated for use in accordance with the invention, these measurements relate to the unencapsulated/pre-encapsulated powder.

The powder particles are preferably at least approximately spherical or spheroidal. However, particles of other shapes such as flakes and filings are also contemplated. They may be formed by any suitable method, for example, filing, milling or grinding, electrolysis of a molten metal salt or precipitation from a solution of a metal salt. The zinc powder is preferably sufficiently pure so as to be materially free of harmful components such as heavy metal contaminants. Preferably the zinc powder consists of at least 99%, for example at least 99.8% zinc by weight.

Copper Powder

Copper powder for use in accordance with the invention is preferably equivalent to as described above in relation to zinc powder.

Organic Acid

The invention comprises any suitable organic acid or mixture of organic acids. Appropriate acids may include 10-hydroxydecanoic acid, acetylsalicylic acid, alphahydroxy acid, glycolic acid, hyaluronic acid, lactic acid, salicylic acid and citric acid. More preferably the acid is succinic acid (especially that derived from amber extract), lactic acid, ferulic acid (especially from a barley extract) or hyaluronic acid. According to certain embodiments the organic acid comprises at least succinic acid, ferulic acid and lactic acid. It is a requirement of the invention that the organic acid reacts with metallic zinc and/or metallic copper in a corrosion reaction.

Encapsulation

The metal powders of the invention are used in encapsulated forms. The encapsulation preferably protects the metal powder from aqueous ingredients of the cosmetic composition. For example, it will protect it from the other ingredients of the cosmetic component(s) in which it is provided. For example, according to the invention, electro-stimulation of the skin cells is provided when corrosion currents are essentially set up on the skin surface when metal powder comes into contact with the organic acid ingredient.

The encapsulation substantially prevents the contact between the metal powder and the organic acid and thereby substantially prevents the corrosion of the metal powder. According to some embodiments “prevents” means reduce the rate of corrosion of the metal powder by more than 90%. According to other embodiments contact between the metal powder of the organic acid may be measured by loss of mass of the metal powder and encapsulation preferably prevents at least 90%, for example at least 99%, of this loss of mass over a 6 month period at room temperature. Clearly, there may be a minor proportion of encapsulated powders wherein the encapsulation is damaged or imperfect. In order to qualify as encapsulated, at least 50% of the powder particles may need to be encapsulated in accordance with the above criteria.

The encapsulation is preferably sufficiently brittle such that it may be functionally removed or ruptured by mechanical shearing as a result of the product being massaged into the skin. Additionally or alternatively the functional integrity of the encapsulation is overcome as a result of the product being massaged into the skin because the skin warms the encapsulation causing it to melt or soften. According to some embodiments, the encapsulation may be functionally degraded by a combination of increased temperature and mechanical disruption.

Preferably the encapsulation comprises a waxy solid, for example a fatty acid ester, for example a monoenoic or dienoic wax ester. Alternatively, it may comprise esters of long chain alcohols or of synthetic waxes such as polyethylene waxes. According to certain preferred embodiments, the encapsulation material comprises stearyl palmitate as its major ingredient. Other ingredients according to certain embodiments include hydrolysed silica, lauryl laurate and hydrolysed corn starch. According to certain embodiments the encapsulation material consists of more than 60%, for example more than 70%, 80% or 90% stearyl palmitate with smaller proportion of hydrolysed silica and optionally up to 2% lauryl laurate and hydrolysed corn starch.

Encapsulation Methods

The metal powder may be encapsulated by any suitable method. Encapsulation techniques are well known to those skilled in the art and include but are not limited to, spray-drying, precipitation onto the particle, spraying of encapsulant, coating in the fluidized bed or use of a rotary granulator. The encapsulation is preferably between 30 and 90% of the total particle (by weight the remainder being the metallic powder) for example between 40 and 80% or 50 and 70%.

Further Features

According to all aspects of the invention, the composition contains one or more of the following ingredients:
purified peptides from Alisma plantago or other plant or yeast oligo peptides.

### EXAMPLES

The invention is further described by reference to the following examples, which are not to be taken as limiting the scope of the claims but which disclose features which may, at the applicant's discretion, be incorporated into the scope of the claims.

### Example 1

### Encapsulation of Copper

Encapsulated copper powder was obtained from Salvona Technologies, Hamilton, N.J., USA. The product is sold under the “MicroSal™ Copper” brand name and the identifier code 8690-02 and has the following composition:

| Component | Minimum w/w % | Maximum w/w % |
|---|---|---|
| Stearyl Palmitate | 33.3 | 100.0 |
| Copper Powder | 1.0 | 33.0 |
| Hydrolysed Silica | 1.0 | 33.0 |
| Lauryl Laurate | 0.0 | 1.0 |
| Hydrolysed Corn Starch | 0.0 | 1.0 |

Particle size of the encapsulated product is between 60 and 200 μm in diameter.

### Example 2

### Encapsulation of Zinc

Encapsulated zinc powder was obtained from Salvona Technologies, Hamilton, N.J., USA. The product is sold under the “MicroSal™ Zinc” brand name and the identifier code 8690-01 and has the following composition:

| Component | Minimum w/w % | Maximum w/w % |
|---|---|---|
| Stearyl Palmitate | 33.3 | 100.0 |
| Zinc Powder | 1.0 | 33.0 |
| Hydrolysed Silica | 1.0 | 33.0 |
| Lauryl Laurate | 0.0 | 1.0 |
| Hydrolysed Corn Starch | 0.0 | 1.0 |

Particle size of the encapsulated product is between 60 and 200 μm in diameter.

### Example 3

### Composition of Energiser Gel

The following composition was prepared in a standard cosmetic gel base. All percentages are given as w/w in the final composition.
5% Somitine™. This is aqueous solution of glycerin and mannosyl glucuronic acid oligoglucan obtained from Greentech Biotechnologies, Saint Beauzire, France.
0.5% encapsulated copper as described in Example 1. Total metallic copper content was 0.085% w/w.
0.5% encapsulated zinc as described in Example 2. Total metallic zinc content was 0.085% w/w.
0.2% amber oil extract obtained from Baltic amber.

### Example 4

### Composition of Day Cream

The following composition was prepared in a day cream cosmetic base. All percentages are given as w/w in the final composition.
0.1% sea salt
0.1% lactic acid
0.1% ferulic acid (standardised in a barley extract)
0.1% succinic acid
0.4% amber oil extract as described in Example 3
3.0% of plant peptide mixture from Alisma plantago-aquatica (CAS No 90320-32-4) from Silab, Saint Viance, France
0.2% cranberry seed oil
2% shea butter
3% coconut oil
0.1% hyaluronic acid
6% almond oil
0.2% sodium PCA.

### Example 5

### Composition of Night Cream

The following composition was prepared in a night cream cosmetic base. All percentages are given as w/w in the final composition.
0.1% sea salt
0.1% lactic acid
0.1% ferulic acid (standardised in a barley extract)
0.1% succinic acid
0.4% amber oil extract as described in Example 3
4% Simulskin™, hydrogenated sunflower fatty acids, Lucas Meyer Cosmetics, Champlan, France
0.2% calcium PCA
0.5% Sepitonic M3™ (magnesium aspartate, zinc gluconate, copper gluconate), Seppic, Paris La Defense, France
4% shea butter
5% almond oil
3% jojoba oil
0.1% hyaluronic acid

### Example 6

### Finished Products 100 ml of each of the energiser composition, the day cream composition and the night cream composition were loaded into pump dispensers.

For a “day product” the dispenser containing the energiser composition and the dispenser containing the day cream composition were packaged into an outer box.

For a “night product” the dispenser containing the energiser composition and the dispenser containing the night cream composition were packaged into an outer box.

### Example 7

### Study of Glucose Consumption

In vitro studies were carried out by Bioalternatives 86160 Gencay, France using their reconstructed human epidermis model. Formulations were applied topically to the skin (at 5mg/cm²) and massaged into the skin surface. The reconstructed epidermis was fed with glucose at lmg/ml and consumption of glucose was measured at 24 hours.

3 Conditions Used
“Control”—Energiser gel as in Example 3 but without the encapsulated metal components.
“Energiser only”—skin treated with Energiser gel composition of Example 3.
“Dual Treatment”—skin treated with Energiser gel composition of Example 3 and the day cream composition of Example 4.

Results

Glucose consumption was measured and the percentage change compared to the control sample calculated thus:

$$Percentage change = \frac{( “ treated value ” - control value ) \times 100}{Control value}$$

| | Consumed glucose in 24 hr |
|---|---|
| Sample | as % increase over control |
| Control | 0% (baseline) |
| “Energiser only” | +16% |
| “Dual Treatment” | +27% |

### Conclusion

The Energiser gel of the invention increases glucose consumption in human epidermis both when applied alone and in combination with a day cream composition containing further organic acids and nutrients.

### Example 8

### Repeat of Example 7 Over Longer Time Period

The experiment of Example 7 was repeated in order to confirm the results obtained in Example 7 and to see if the increase in uptake of glucose could be sustained beyond 24 hours.

Results

| | Increase in | Increase in | Increase in | Total extra |
|---|---|---|---|---|
| | consumed | consumed | consumed | glucose con- |
| | glucose at | glucose at | glucose at | sumed over |
| | 24 hr (% | 24-48 hrs (% | 48-72 hrs (% | 3 days (% |
| Sample | of control) | of control) | of control) | of control) |
| Control | 0 | 0 | 0 | 0 |
| “Energiser only” | 15.88 | 25.36 | 7.32 | 48.56 |
| “Dual Treatment” | 26.50 | 23.01 | 5.98 | 55.49 |

Conclusion

The data is similar to that reported in Example 7 and supports the conclusions drawn from that Example. It further demonstrates that the increased uptake of glucose brought about by the invention can be sustained.

According to the invention, there are provided products and methods providing electro-stimulation of human epidermal cells. That electro-stimulation may be assayed by measuring increased cell metabolism, for example by measuring increased cellular glucose consumption by a method as described above. Alternatively, an alternative assay may be used to determine successful electro-stimulation including, but not limited to, other measures of cellular metabolic rate, protein synthesis, cell turnover or skin appearance.

1. A cosmetic product comprising, in one or more cosmetic components each comprising a cosmetic base:
zinc powder and/or copper powder; and
an organic acid,
wherein the zinc powder and/or copper powder are encapsulated with a hydrophobic encapsulation which prevents the organic acid coming into contact with the zinc powder and/or copper powder.

2. A cosmetic product according to claim 1, wherein the zinc powder and/or copper powder is encapsulated in a brittle waxy solid.

3. A cosmetic product according to claim 2, wherein the brittle waxy solid comprises stearyl palmitate as its major ingredient.

4. A cosmetic product according to claim 3, wherein the organic acid is selected from 10-hydroxydecanoic acid, acetyl salicylic acid, alphahydroxy acid, glycolic acid, succinic acid, lactic acid, ferulic acid and hyaluronic acid and mixtures thereof.

5. A cosmetic product according to claim 4, wherein the organic acid comprises succinic acid or ferulic acid or a mixture of both.

6. A cosmetic product according to claim 5, wherein the product is capable of providing electro-stimulation of human epidermal cells when applied thereto.

7. A method of applying a cosmetic product for the skin comprising:
applying a first component of the product to the skin;
applying a second component of the product to the skin;
massaging the skin to mix the first and second components together, characterised in that one or both components contains an organic acid and one or both components contain zinc powder and/or copper powder which has been encapsulated with a hydrophobic encapsulation, such that, when the skin is massaged to mix the first and second components together, the hydrophobic encapsulation is ruptured allowing the zinc powder and/or copper powder to come into contact with the organic acid.

8. A method as claimed in claim 7 wherein the zinc powder and/or copper powder is encapsulated in a brittle waxy solid.

9. A method of improving the cosmetic appearance of skin by boosting metabolic rate of epidermal cells comprising:
applying one or more compositions to the surface of the skin;
wherein the one or more compositions collectively comprise an organic acid in an aqueous cosmetic base and zinc powder and/or copper powder, wherein the zinc powder and/or copper powder is encapsulated in a hydrophobic encapsulation such that the zinc powder and/or copper powder is wholly or partly prevented from reacting with the organic acid;
massaging the skin surface to break the encapsulation and allow the zinc powder and/or copper powder to react more fully with the organic acid.

10. A method according to claim 9, wherein the zinc powder and/or copper powder is encapsulated in a brittle waxy solid.

11. A method according to claim 10, wherein the brittle waxy solid comprises stearyl palmitate as its major ingredient.

12. A method according to claim 9, wherein the organic acid is selected from 10-hydroxydecanoic acid, acetyl salicylic acid, alphahydroxy acid, glycolic acid, succinic acid, lactic acid, ferulic acid and hyaluronic acid and mixtures thereof.

13. A method according to claim 12, wherein the organic acid comprises succinic acid or ferulic acid or a mixture of both.

14. A cosmetic product according to claim 1, wherein the organic acid is selected from 10-hydroxydecanoic acid, acetyl salicylic acid, alphahydroxy acid, glycolic acid, succinic acid, lactic acid, ferulic acid and hyaluronic acid and mixtures thereof.

15. A cosmetic product according to claim 14, wherein the organic acid comprises succinic acid or ferulic acid or a mixture of both.

16. A cosmetic product according to claim 2, wherein the organic acid is selected from 10-hydroxydecanoic acid, acetyl salicylic acid, alphahydroxy acid, glycolic acid, succinic acid, lactic acid, ferulic acid and hyaluronic acid and mixtures thereof.

17. A cosmetic product according to claim 16, wherein the organic acid comprises succinic acid or ferulic acid or a mixture of both.

18. A method as claimed in claim 8, wherein the brittle waxy solid comprises stearyl palmitate as its major ingredient.

19. A method as claimed in claim 18, wherein the organic acid is selected from 10-hydroxydecanoic acid, acetyl salicylic acid, alphahydroxy acid, glycolic acid, succinic acid, lactic acid, ferulic acid and hyaluronic acid and mixtures thereof.

20. A method as claimed in claim 19, wherein the organic acid comprises succinic acid or ferulic acid or a mixture of both.
